(19) United States
(12) Patent Application Publication
(10) Pub. No.: US 2022/0331445 A1
(43) Pub. Date: Oct. 20, 2022

(54) HIGH DENSITY LIPOPROTEIN-LIKE NANOPARTICLES AS INDUCERS OF FERROPTOSIS IN CANCER

(71) Applicant: Northwestern University, Evanston, IL (US)

(72) Inventors: C. Shad Thaxton, Chicago, IL (US); Jonathan S. Rink, Park Ridge, IL (US); Leo I. Gordon, Winnetka, IL (US)

(73) Assignee: Northwestern University, Evanston, IL (US)

(21) Appl. No.: 17/760,990

(22) PCT Filed: Sep. 18, 2020
(86) PCT No.: PCT/US2020/051549

§ 371 Date: Mar. 16, 2022

Related U.S. Application Data
(60) Provisional application No. 62/902,342, filed on Sep. 18, 2019.

(51) Int. Cl. A61K 47/69; A61P 35/00; B82Y 5/00; A61K 33/242; A61K 9/51; A61K 47/54

(52) U.S. Cl. CPC A61K 47/6929; A61P 35/00; B82Y 5/00; A61K 33/242; A61K 9/5123; A61K 47/544

(57) ABSTRACT

Disclosed herein are compositions and methods for treating a subject having cancer and other ferroptosis disorders with high density lipoprotein-like nanoparticles that induce ferroptosis.

### CROSS-REFERENCE TO RELATED APPLICATIONS

This application claims the benefit under 35 U.S.C. § 119(e) of the filing date of U.S. Patent Application Ser. No. 62/902,342, filed Sep. 18, 2019. The contents of the above-referenced application is hereby incorporated by reference herein in its entirety.

### BACKGROUND OF THE INVENTION

Cancer is the second leading cause of death in the USA and globally. Finding a targeted therapeutic with efficacy across multiple malignancies offers incredible potential value, both in improving patient outcomes and from an economic standpoint. Despite long-term remission observed in some patients with lymphoma, greater than one third of patients with the most common subtype, diffuse large B cell lymphoma (DLBCL), will relapse or have disease that is refractory to primary treatment (1-3). This is especially the case for patients in high-risk groups identified by molecular and clinical prognostic factors (4,5). Experimental therapies for these patients, including immunotherapy and cell-based therapies, have modest success rates, high cost, and toxicity.

### SUMMARY OF THE INVENTION

The present disclosure is based, at least in part, on compositions, kits, and methods for treating a subject having cancer by administering a high density lipoprotein nanoparticle (HDL-NP) that targets malignant cells and induces ferroptosis.

Accordingly, one aspect of the present disclosure provides a method of treating a subject having cancer by administering to the subject a synthetic nanostructure comprising a nanostructure core, a shell comprising a lipid surrounding and attached to the nanostructure core, wherein the shell comprises a phospholipid; wherein the subject has cancer cells and wherein the synthetic nanostructure is administered in an effective amount to induce ferroptosis in the cancer cells.

Another aspect of the present disclosure provides a method of reducing, in a population of cells, the number of cancer cells, the method comprising contacting the cancer cells with a synthetic nanostructure comprising: a nanostructure core, a shell comprising a lipid surrounding and attached to the nanostructure core, wherein the shell comprises a phospholipid; wherein the synthetic nanostructure is in an effective amount to induce ferroptosis in the cancer cells.

In some embodiments of the present disclosure the nanostructure core is Ag, Au, Pt, Fe, Cr, Co, Ni, Cu, Zn, and other transition metals, a semiconductor (e.g., silicon, silicon compounds and alloys, cadmium selenide, cadmium sulfide, indium arsenide, and indium phosphide), or an insulator (e.g., ceramics such as silicon oxide).

In some embodiments, the synthetic nanostructure further comprises an apolipoprotein. In some embodiments, the apolipoprotein is apolipoprotein A-I, apolipoprotein A-II, or apolipoprotein E.

In some embodiments, the synthetic nanostructure further comprises a cholesterol.

In some embodiments, the phospholipid shell comprises a lipid monolayer.

In some embodiments, the phospholipid shell comprises a lipid bilayer. In some embodiments, at least a portion of the lipid bilayer is covalently bound to the nanostructure core.

In some embodiments, the nanostructure core has a largest cross-sectional dimension of less than or equal to about 500 nanometers (nm). In some embodiments, the nanostructure core has a largest cross-sectional dimension of less than or equal to about 250 nanometers (nm). In some embodiments, the nanostructure core has a largest cross-sectional dimension of less than or equal to about 100 nanometers (nm). In some embodiments, the nanostructure core has a largest cross-sectional dimension of less than or equal to about 75 nanometers (nm). In some embodiments, the nanostructure core has a largest cross-sectional dimension of less than or equal to about 50 nanometers (nm). In some embodiments, the nanostructure core has a largest cross-sectional dimension of less than or equal to about 30 nanometers (nm). In some embodiments, the nanostructure core has a largest cross-sectional dimension of less than or equal to about 15 nanometers (nm). In some embodiments, the nanostructure core has a largest cross-sectional dimension of less than or equal to about 10 nanometers (nm). In some embodiments, the nanostructure core has a largest cross-sectional dimension of less than or equal to about 5 nanometers (nm). In some embodiments, the nanostructure core has a largest cross-sectional dimension of less than or equal to about 3 nanometers (nm).

In some embodiments, the nanostructure core has an aspect ratio of greater than about 1:1. In some embodiments, the nanostructure core has an aspect ratio of greater than 3:1. In some embodiments, the nanostructure core has an aspect ratio of greater than 5:1.

In some embodiments, the phospholipid comprise 1,2-dipalmitoyl-sn-glycero-3-phosphocholine (DPPC), 1,2-dioleoyl-sn-glycero-3-phosphocholine (DOPC), 1,2-dipalmitoyl-sn-glycero-3-phosphoethanolamine (16:0 PE), 1,2-distearoyl-sn-glycero-3-phosphoethanolamine (18:0 PE), sphingomyelin, 1,2-dioleoyl-3-trimethylammonium-propane (DOTAP), or a combination thereof.

In some embodiments, the subject has been diagnosed with cancer. In some embodiments, the subject has been diagnosed with a ferroptosis sensitive malignancy or cholesterol auxotrophic malignancy. In some embodiments, the cancer is selected from: B-cell lymphoma, renal cell carcinoma, T-cell lymphoma, gastric cancer, ovarian cancer, endometrial adenocarcinoma sarcoma, anaplastic large cell lymphoma, clear cell renal cell carcinoma (ccRCC), platinum resistant ovarian cancer, and clear cell ovarian cancer.

In some embodiments, the synthetic nanostructure is administered to the subject or contacted to the cells more than once. In some embodiments, the synthetic nanostructure is administered to the subject or contacted to the cells at least once per month. In some embodiments, the synthetic nanostructure is administered to the subject or contacted to the cells at least once per week. In some embodiments, the synthetic nanostructure is administered to the subject or contacted to the cells at least once per day. In some embodiments, the synthetic nanostructure is administered to the subject or contacted to the cells twice per day.

In some embodiments, any of the methods of the disclosure further comprise administering to the subject a ferroptosis inducer compound.

In some embodiments, any of the methods of the disclosure further comprise determining if the cancer is sensitive to ferroptosis.

In some aspects, the disclosure relates to a method of treating a subject having a ferroptosis sensitive disorder comprising: identifying a subject having a ferroptosis sensitive disorder; and administering to the subject a synthetic nanostructure comprising: a nanostructure core, a shell comprising a lipid surrounding and attached to the nanostructure core, wherein the shell comprises a phospholipid; in an effective amount to induce ferroptosis in diseased cells of the subject.

In some aspects, the disclosure relates to a composition comprising synthetic nanostructure comprising: a nanostructure core, a shell comprising a lipid surrounding and attached to the nanostructure core, wherein the shell comprises a phospholipid and a ferroptosis inducer compound.

In some aspects, the disclosure relates to a method for inducing ferroptosis in a cell, comprising: identifying a cell as being a ferroptosis sensitive cell, and contacting the cell with a nanostructure core, a shell comprising a lipid surrounding and attached to the nanostructure core, wherein the shell comprises a phospholipid in an effective amount to induce ferroptosis in the cell.

In some embodiments, the subject of any of the methods of the disclosure is a mammal. In some embodiments, the subject of any of the methods of the disclosure is human.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. In the drawings:

FIG. 1 includes bar graphs showing that HDL NPs down-regulate GPX4 (*p<0.05 at dosages of 20 nM and 50 nM vs 0 nm) in both Ramos and SUDHL4 cells.

FIG. 2 includes a bar graph showing that HDL NPs induce Ferroptosis in SUDHL4 (Diffuse Large B Cell Lymphoma) cells (*p<0.05 vs Control (PBS)).

FIG. 3 includes a bar graph showing that HDL NPs induce Ferroptosis in Ramos (Burkitt's Lymphoma) cells (*p<0.05 vs Control (PBS)).

FIGS. 4A-4B include plots showing that HDL NPs induce lipid peroxide accumulation in SUDHL4 cells (*p<0.05 vs 0 hrs).

FIGS. 5A-5B include plots showing that HDL NPs induce lipid peroxide accumulation in Ramos cells (*p<0.05 vs 0 hrs).

FIGS. 6A-6B include plots showing that SR-B1 expression and HDL NP efficacy in cholesterol auxotrophic cell lines (*p<0.05 vs PBS). SNU-1: Gastric cancer. SUDHL1: ALK+Anaplastic Large T Cell Lymphoma. SR: ALK+Anaplastic Large T Cell Lymphoma. U937: Histiocytic lymphoma. HEC-1B: Endometrial adenocarcinoma. U266B1: Myeloma. Ramos: Burkitt's lymphoma (positive control). Jurkat: T cell lymphoma (negative control).

FIGS. 7A-7B include plots showing SUDHL1 tumor xenograft models of tumor volumes, weights and in vivo GPX4 expression (*p=0.0339 and **p=0.0238).

FIG. 8 includes plots showing SUDHL1 in vivo Ferroptosis assay (n=9 for PBS, 10 for HDL NPs, *p=0.0006).

FIG. 9 includes a bar graph showing that HDL NPs induce Ferroptosis in 786-O (renal cell carcinoma-clear cell) cells (*p<0.05 vs HDL NPs+Ferrostatin-1 and HDL NPs+DFO).

FIG. 10 includes a bar graph showing that HDL NPs induce Ferroptosis in Caki-2 (renal cell carcinoma-papillary) cells (*p<0.05 vs HDL NPs+Ferrostatin-1 and HDL NPs+DFO).

FIGS. 11A-11B include plots showing that HDL NPs induce lipid peroxide accumulation in 786-O and Caki-2 cells (*p=0.0096 and **p=0.0011).

FIGS. 12A-12G show results generated in 786-O Cell Line, renal cell carcinoma cell line. FIG. 12A shows that siRNA knockdown of SR-B1 downregulates GPX4 expression. Data is shown at 96 and 120 hours (hr). 25 μg of protein, SR-B1 antibody Abcam (ab52629, 1:2000), GPX4 Abcam (ab41787, 1:20,000), Beta actin Cell Signaling (13E5, 1:2,000). FIG. 12B shows that siRNA knockdown of SR-B1 downregulation induces cell death. FIG. 12C shows Western Blots of GPX4 and SR-B1 over various time courses with varying concentrations of HDL NPs. As can be seen, HDL NPs do not directly regulate SR-B1 receptor expression; however, HDL NPs drastically downregulate GPX4 expression in a time and dose (e.g., HDL NP concentration) dependent manner. FIG. 12D shows Western Blots illustrating that HDL NPs drastically downregulate GPX4 expression in the presence of Sutent. 8 μg protein, GPX4 Abcam (ab41787, 1:5,000), Beta actin Cell Signaling (13E5, 1:2,000). FIG. 12E shows HDL NPs increase the expression of oxidized lipids. FIG. 12F shows an MTS Rescue Assay; cell death induced by HDL NPs is rescued by ferrostatin-1 and deferoxamine. FIG. 12G shows in vivo data of HDL NPs reducing 786-O tumor burden (upper left panel); HDL NPs increase survival (upper right panel); and HDL NPs increase oxidized lipids in tumors after 5 treatments of the HDL NP (bottom middle panel).

FIGS. 13A-13B shows results generated from HDL NPs in 769-P, a clear cell renal carcinoma cell line. FIG. 13A shows Western Blots of GPX4 and HDL NP downregulate thereof. FIG. 13B MTS data showing cell death induced by HDL NPs is rescued by ferrostatin-1 and deferoxamine.

FIGS. 14A-14D shows results generated from HDL NPs in OVCAR5 cell line, a platinum sensitive ovarian cancer cell line. FIG. 14A shows a Western Blot of GPX4 illustrating HDL NPs downregulate GPX4 expression. FIG. 14B C11-BODIPY Flow Data illustrating that HDL NPs increase the expression of oxidized lipids. FIG. 14C shows MTS assay cell death induced by HDL NPs is rescued by ferrostatin-1 and deferoxamine. FIG. 14D shows Western Blots of SR-B1 and GPX4 and that siRNA knockdown of SR-B1 downregulates GPX4 expression.

FIGS. 15A-15C shows results generated from HDL NPs in OVCAR5 CP Resistant Cell Line, a platinum resistant ovarian cancer cell line. FIG. 15A shows a Western Blot of GPX4 and that HDL NPs downregulate GPX4 expression. FIG. 15B shows an MTS assay of cell death induced by HDL NPs is rescued by ferrostatin-1 and deferoxamine. FIG. 15C shows HDL NPs increase the expression of oxidized lipids.

FIG. 16 shows results generated from HDL NPs in ES2 cell line, a clear cell ovarian carcinoma cell line. Western Blot are shown of GPX4 and that HDL NPs downregulate GPX4 expression.

### DETAILED DESCRIPTION OF THE INVENTION

The present invention relates to drugs comprising high density lipoprotein-like nanoparticles (HDL NPs) that are useful for treating subjects with cancer and other disorders. The drugs target cancerous malignant cells and cause targeted cell death.

Altered metabolism is a hallmark of cancer, with malignant cells requiring increasing quantities of various nutrients, including cholesterol and cholesteryl esters. While promoting cellular proliferation, this altered metabolic state can also sensitize the cell to an iron- and oxygen-dependent necroptotic form of programmed cell death called ferroptosis. The present disclosure provides compositions and method of using bio-inspired high density lipoprotein-like nanoparticles (HDL NPs; also referred to as synthetic nanostructures or cholesterol-poor high-density lipoprotein (HDL)-like nanoparticles), which mimics the size, surface composition, and shape of natural HDLs.

The HDL NPs of the present invention bind to the receptor for mature HDLs, scavenger receptor type B1 (SR-B1), also referred to as SCARB1, which are used interchangeably herein (a high-affinity receptor for cholesterol-rich high-density lipoproteins (HDL)), and starve the malignant cells of cholesterol by preventing internalization of cholesteryl esters from natural HDLs and effluxing free cholesterol from the cell. It has been discovered that HDL NPs can effectively induce ferroptosis in susceptible cells, and, for instance, that HDL NP therapy targeting SCARB1 induced lymphoma cell death through a mechanism involving GPX4 and ferroptosis. Initially, the data (presented in the patent application) revealed that HDL NPs obligate cellular expression of de novo cholesterol biosynthesis genes, which is accompanied by reduced GPX4 expression. It was further shown that reduced GPX4 expression leads to an increase in membrane oxidized lipids and cell death through a mechanism consistent with ferroptosis in cell lines, in an in vivo xenograft model, and in primary samples obtained from patients with B cell lymphoma.

Ferroptosis is an oxygen- and iron-dependent form of necroptosis characterized by accumulation of cell membrane lipid and cholesterol peroxides that results from the targeted inhibition of the lipid hydroperoxidase glutathione peroxidase 4 (GPX4). Cells become vulnerable to ferroptosis after GPX4 inhibition because the enzyme reduces and detoxifies lipid peroxides (L-OOH) by converting them to corresponding lipid alcohols (L-OH). Malignant cells under oxidative stress are significantly more sensitive to ferroptosis because of higher levels of reactive oxygen species and a reliance on GPX4 activity to mitigate toxic L-OOH accumulation. Small molecule inhibitors of GPX4 have been developed and tested, but they are toxic and lack specificity, which limits in vivo use and clinical relevance.

The cholesterol-poor HDL NP targets SCARB1 in cholesterol uptake dependent lymphoma cells and other susceptible cells. HDL NP binding to SCARB1 results in a switch from a baseline dependence on cholesterol uptake and high GPX4 expression, to one favoring de novo cholesterol biosynthesis, which is accompanied by reduced expression of GPX4. As GPX4 is absolutely required by the cancer cell to reduce the burden of membrane lipid peroxides, this metabolic switch leaves the cancer cell particularly vulnerable. Accordingly, an increase in the accumulation of oxidized membrane lipids leads to cell death through a mechanism of ferroptosis.

The methods disclosed herein are useful, in some aspects, for inducing ferroptosis in a ferroptosis sensitive cell. Recent studies have shown that ferroptosis is closely related to the pathophysiological processes of many diseases, such as tumors, nervous system diseases, ischemia-reperfusion injury, kidney injury, and blood diseases. A ferroptosis sensitive cell is a cell which has an iron dependence and can undergo programmed cell death, with resulting lipid peroxide accumulation and cell death. In some embodiments the ferroptosis cell is a tumor cell, such as a pancreatic cancer, hepatocellular carcinoma (HCC), gastric cancer, colorectal cancer, breast cancer, lung cancer, clear cell renal cell carcinoma (ccRCC), adrenocortical carcinomas, ovarian cancer, head and neck cancer, and melanoma.

In addition to cancer, HDL NPs are useful for inhibiting ferroptosis in neurological diseases such as traumatic brain injury, stroke, neurodegenerative disorders such as Huntington's disease, Parkinson's disease, ALS, and Friedreich's ataxia, acute kidney disease or injury.

Methods for determining sensitivity to ferroptosis are known. For instance, knowledge about the role of NAD(P)H in the various pathways can be used to predict sensitivity to ferroptosis. Additionally, the expression levels of FSP have a positive correlation with ferroptosis resistance in cells and can be used to detect sensitivity, particularly in cancer cells. The expression of FSP1 has been used for predicting the efficacy of ferroptosis-inducing drugs in cancers and for identifying potential ferroptosis inducers.

The inventors found that HDL NPs potently induce ferroptosis in a wide range of malignancies, including ferroptosis sensitive malignancies (B-cell lymphomas, renal cell carcinoma) and cholesterol auxotrophic malignancies (T-cell lymphomas, gastric cancer, endometrial adenocarcinoma). DLBCL (diffuse large B cell lymphoma) is a cancer type particularly sensitive to cell death by ferroptosis. In some embodiments of the present invention, HDL NPs are synthesized by surface functionalizing a gold nanoparticle core (optionally 5 nm) with the HDL-defining apolipoprotein A1 (ApoA1) and a phospholipid bilayer. Once assembled, these nanoparticles mimic the surface composition, size, and shape of mature, cholesteryl ester rich HDLs; however, they are a poor source of cholesterol, given the presence of the gold nanoparticle core occupying the real estate (e.g., position and volume in the HDL NP) typically reserved for cholesteryl esters. By binding to the receptor for mature HDLs and preventing cholesteryl ester uptake, HDL NPs induce a state of cholesterol starvation, which leads to induction of ferroptosis in B-cell lymphomas (diffuse large B cell lymphoma, Burkitt's lymphoma), T-cell lymphomas (anaplastic large cell lymphoma), renal cell carcinomas (clear cell and papillary), gastric cancer and endometrial adenocarcinoma.

The HDL NPs of the present invention exploit the metabolic state of malignant cells. They exhibit preferential targeting and high efficacy against malignant cells compared to normal, healthy cells. The efficacy against the malignant cells is determined by the metabolic profile of the cells, rather than the cell of origin. The HDL NPs effectively reduce cancer cell viability, shrink malignant tumors, and activate host immune cells against the malignant cells. This allows cells of various origins to be targeted and treatment of a wide range of cancers. Additionally, the compositions of the present invention exhibit better biodistribution and pharmacokinetics than other ferroptosis inducers (e.g. small molecule inhibitors).

Cancer

In some embodiments, the compositions of the present invention can be used to treat or prevent cancer. In some embodiments, the cancer is characterized by cells that express scavenger receptor class B type 1 (SR-B1).

Non-limiting examples of cancers include: bladder cancer, breast cancer, colon and rectal cancer, endometrial cancer, kidney or renal cell cancer, leukemia, lung cancer, melanoma, Non-Hodgkin lymphoma, pancreatic cancer, prostate cancer, ovarian cancer, stomach cancer, wasting disease, and thyroid cancer. Additional non-limiting examples of cancer include Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, soft tissue Ewing's sarcoma, soft tissue sarcoma, synovial sarcoma, malignant lymphoma (reticulum cell sarcoma), multiple myeloma, malignant giant cell tumor chordoma, desmoid-type fibromatosis, fibroblastic sarcoma, gastrointestinal stromal tumors, retroperitoneal sarcoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defomians), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); gynaecological sarcoma, Kaposi's sarcoma, peripheral never sheath tumor, Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, SertoliLeydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles, dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

As used herein, the terms “disease” and “disorder” refer to any condition that would benefit from treatment with a composition of the present invention (e.g., any of the compositions or methods as described herein). This includes chronic and acute disorders or diseases including those pathological conditions that predispose the mammal to the disorder in question.

Synthetic Nanostructures

In some embodiments of the present disclosure a subject having cancer is treated by administering a synthetic nanostructure as described herein. The synthetic nanostructure comprises a nanostructure core, a shell, the shell comprising a lipid layer surrounding and attached to the nanostructure core. In some embodiments, the synthetic nanostructure further comprises a protein associated with the shell. Examples of synthetic nanostructures useful for the present purposes are described below.

Examples of synthetic nanostructures that can be used in the methods are described herein. The structure (e.g., synthetic nanostructure, HDL NP) has a core and a shell surrounding the core. In embodiments in which the core is a nanostructure, the core includes a surface to which one or more components can be optionally attached. For instance, in some cases, a core is a nanostructure surrounded by shell, which shell includes an inner surface and an outer surface. The shell may be formed, at least in part, of one or more components, such as a plurality of lipids, which may optionally associate with one another and/or with surface of the core. For example, components may be associated with the core by being covalently attached to the core, physiosorbed, chemisorbed, or attached to the core through ionic interactions, hydrophobic and/or hydrophilic interactions, electrostatic interactions, van der Waals interactions, or combinations thereof. In one particular embodiment, the core includes a gold nanostructure and the shell is attached to the core through a gold-thiol bond.

Optionally, components can be crosslinked to one another. Crosslinking of components of a shell can, for example, allow the control of transport of species into the shell, or between an area exterior to the shell and an area interior of the shell. For example, relatively high amounts of crosslinking may allow certain small, but not large, molecules to pass into or through the shell, whereas relatively low or no crosslinking can allow larger molecules to pass into or through the shell. Additionally, the components forming the shell may be in the form of a monolayer or a multilayer, which can also facilitate or impede the transport or sequestering of molecules. In one exemplary embodiment, shell includes a lipid bilayer that is arranged to sequester cholesterol and/or control cholesterol efflux out of cells, as described herein.

It should be understood that a shell that surrounds a core need not completely surround the core, although such embodiments may be possible. For example, the shell may surround at least 50%, at least 60%, at least 70%, at least 80%, at least 90%, or at least 99% of the surface area of a core. In some cases, the shell substantially surrounds a core. In other cases, the shell completely surrounds a core. The components of the shell may be distributed evenly across a surface of the core in some cases, and unevenly in other cases. For example, the shell may include portions (e.g., holes) that do not include any material in some cases. If desired, the shell may be designed to allow penetration and/or transport of certain molecules and components into or out of the shell, but may prevent penetration and/or transport of other molecules and components into or out of the shell. The ability of certain molecules to penetrate and/or be transported into and/or across a shell may depend on, for example, the packing density of the components forming the shell and the chemical and physical properties of the components forming the shell. As described herein, the shell may include one layer of material, or multilayers of materials in some embodiments.

In certain embodiments that synthetic nanostructure may further include one or more agents, such as a therapeutic or diagnostic agent. The agent may be a diagnostic agent (which may also be known as an imaging agent), a therapeutic agent, or both a diagnostic agent and a therapeutic agent. In certain embodiments the diagnostic agent is a tracer lipid. Tracer lipids may comprise a chromophore, a biotin subunit, or both a chromophore and a biotin subunit. The synthetic nanostructures (e.g. HDL NPs) can also be functionalized with other types of cargo such as nucleic acids. In certain embodiments the therapeutic agent may be a nucleic acid, antiviral agent, antineurological agent, or antirheumatologic agent.

The one or more agents may be associated with the core, the shell, or both; e.g., they may be associated with surface of the core, inner surface of the shell, outer surface of the shell, and/or embedded in the shell. For example, one or more agents may be associated with the core, the shell, or both through covalent bonds, physisorption, chemisorption, or attached through ionic interactions, hydrophobic and/or hydrophilic interactions, electrostatic interactions, van der Waals interactions, or combinations thereof.

In some cases, the synthetic nanostructure is a synthetic cholesterol binding nanostructure having a binding constant for cholesterol, Kd. In some embodiments, Kd is less than or equal to about 100 μM, less than or equal to about 10 μM, less than or equal to about 1 μM, less than or equal to about 0.1 μM, less than or equal to about 10 nM, less than or equal to about 7 nM, less than or equal to about 5 nM, less than or equal to about 2 nM, less than or equal to about 1 nM, less than or equal to about 0.1 nM, less than or equal to about 10 pM, less than or equal to about 1 pM, less than or equal to about 0.1 pM, less than or equal to about 10 fM, or less than or equal to about 1 fM. Methods for determining the amount of cholesterol sequestered and binding constants are known in the art.

The core of the nanostructure may have any suitable shape and/or size. For instance, the core may be substantially spherical, non-spherical, oval, rod-shaped, pyramidal, cube-like, disk-shaped, wire-like, or irregularly shaped. In preferred embodiments of the present invention, the core is less than or equal to about 5 nm in diameter. The core (e.g., a nanostructure core or a hollow core) may have a largest cross-sectional dimension (or, sometimes, a smallest cross-section dimension, or diameter) of, for example, less than or equal to about 500 nm, less than or equal to about 250 nm, less than or equal to about 100 nm, less than or equal to about 75 nm, less than or equal to about 50 nm, less than or equal to about 40 nm, less than or equal to about 35 nm, less than or equal to about 30 nm, less than or equal to about 25 nm, less than or equal to about 20 nm, less than or equal to about 15 nm, less than or equal to about 10 nm, less than or equal to about 5 nm, less than or equal to about 4 nm, less than or equal to about 3 nm, less than or equal to about 2 nm, or less than or equal to about 1 nm. In some cases, the core has an aspect ratio of greater than about 1:1, greater than 3:1, or greater than 5:1. As used herein, “aspect ratio” refers to the ratio of a length to a width, where length and width measured perpendicular to one another, and the length refers to the longest linearly measured dimension.

In embodiments in which core includes a nanostructure core, the nanostructure core may be formed from any suitable material. In preferred embodiments, the core is formed from gold (e.g. made of gold (Au)). In some embodiments, the core is formed of a synthetic material (e.g., a material that is not naturally occurring, or naturally present in the body). In one embodiment, a nanostructure core comprises or is formed of an inorganic material. The inorganic material may include, for example, a metal (e.g., Ag, Au, Pt, Fe, Cr, Co, Ni, Cu, Zn, and other transition metals), a semiconductor (e.g., silicon, silicon compounds and alloys, cadmium selenide, cadmium sulfide, indium arsenide, and indium phosphide), or an insulator (e.g., ceramics such as silicon oxide). The inorganic material may be present in the core in any suitable amount, e.g., at least 1 wt %, 5 wt %, 10 wt %, 25 wt %, 50 wt %, 75 wt %, 90 wt %, or 99 wt %. In one embodiment, the core is formed of 100 wt % inorganic material. The nanostructure core may, in some cases, be in the form of a quantum dot, a carbon nanotube, a carbon nanowire, or a carbon nanorod. In some cases, the nanostructure core comprises, or is formed of, a material that is not of biological origin. In some embodiments, a nanostructure includes or may be formed of one or more organic materials such as a synthetic polymer and/or a natural polymer. Examples of synthetic polymers include non-degradable polymers such as polymethacrylate and degradable polymers such as polylactic acid, polyglycolic acid and copolymers thereof. Examples of natural polymers include hyaluronic acid, chitosan, and collagen.

Furthermore, a shell of a structure can have any suitable thickness. For example, the thickness of a shell may be at least 10 Angstroms, at least 0.1 nm, at least 1 nm, at least 2 nm, at least 5 nm, at least 7 nm, at least 10 nm, at least 15 nm, at least 20 nm, at least 30 nm, at least 50 nm, at least 100 nm, or at least 200 nm (e.g., from the inner surface to the outer surface of the shell). In some cases, the thickness of a shell is less than 200 nm, less than 100 nm, less than 50 nm, less than 30 nm, less than 20 nm, less than 15 nm, less than 10 nm, less than 7 nm, less than 5 nm, less than 3 nm, less than 2 nm, or less than 1 nm (e.g., from the inner surface to the outer surface of the shell). Such thicknesses may be determined prior to or after sequestration of molecules as described herein.

Those of ordinary skill in the art are familiar with techniques to determine sizes of structures and particles. Examples of suitable techniques include dynamic light scattering (DLS) (e.g., using a Malvern Zetasizer instrument), transmission electron microscopy, scanning electron microscopy, electroresistance counting and laser diffraction. Other suitable techniques are known to those of ordinary skill in the art. Although many methods for determining sizes of nanostructures are known, the sizes described herein (e.g., largest or smallest cross-sectional dimensions, thicknesses) refer to ones measured by dynamic light scattering.

The shell of a structure described herein may comprise any suitable material, such as a hydrophobic material, a hydrophilic material, and/or an amphiphilic material. Although the shell may include one or more inorganic materials such as those listed above for the nanostructure core, in many embodiments the shell includes an organic material such as a lipid or certain polymers. The components of the shell may be chosen, in some embodiments, to facilitate the sequestering of cholesterol or other molecules. For instance, cholesterol (or other sequestered molecules) may bind or otherwise associate with a surface of the shell, or the shell may include components that allow the cholesterol to be internalized by the structure. Cholesterol (or other sequestered molecules) may also be embedded in a shell, within a layer or between two layers forming the shell.

The components of a shell may be charged, e.g., to impart a charge on the surface of the structure, or uncharged. In some embodiments, the surface of the shell may have a zeta potential of greater than or equal to about −75 mV, greater than or equal to about −60 mV, greater than or equal to about −50 mV, greater than or equal to about −40 mV, greater than or equal to about −30 mV, greater than or equal to about −20 mV, greater than or equal to about −10 mV, greater than or equal to about 0 mV, greater than or equal to about 10 mV, greater than or equal to about 20 mV, greater than or equal to about 30 mV, greater than or equal to about 40 mV, greater than or equal to about 50 mV, greater than or equal to about 60 mV, or greater than or equal to about 75 mV. The surface of the shell may have a zeta potential of less than or equal to about 75 mV, less than or equal to about 60 mV, less than or equal to about 50 mV, less than or equal to about 40 mV, less than or equal to about 30 mV, less than or equal to about 20 mV, less than or equal to about 10 mV, less than or equal to about 0 mV, less than or equal to about −10 mV, less than or equal to about −20 mV, less than or equal to about −30 mV, less than or equal to about −40 mV, less than or equal to about −50 mV, less than or equal to about −60 mV, or less than or equal to about −75 mV. Other ranges are also possible. Combinations of the above-referenced ranges are also possible (e.g., greater than or equal to about −60 mV and less than or equal to about −20 mV). As described herein, the surface charge of the shell may be tailored by varying the surface chemistry and components of the shell.

In one set of embodiments, a structure described herein or a portion thereof, such as a shell of a structure, includes one or more natural or synthetic lipids or lipid analogs (i.e., lipophilic molecules). One or more lipids and/or lipid analogues may form a single layer or a multi-layer (e.g., a bilayer) of a structure. In some instances where multi-layers are formed, the natural or synthetic lipids or lipid analogs interdigitate (e.g., between different layers). Non-limiting examples of natural or synthetic lipids or lipid analogs include fatty acyls, glycerolipids, glycerophospholipids, sphingolipids, saccharolipids and polyketides (derived from condensation of ketoacyl subunits), and sterol lipids and prenol lipids (derived from condensation of isoprene subunits).

In one particular set of embodiments, a structure described herein includes one or more phospholipids. The one or more phospholipids may include, for example, phosphatidylcholine, phosphatidylglycerol, lecithin, (3, γ-dipalmitoyl-α-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, 1,2-dipalmitoyl-sn-glycero-3-phosphothioethanol, and combinations thereof. In some cases, a shell (e.g., a bilayer) of a structure includes 50-200 natural or synthetic lipids or lipid analogs (e.g., phospholipids). For example, the shell may include less than about 500, less than about 400, less than about 300, less than about 200, or less than about 100 natural or synthetic lipids or lipid analogs (e.g., phospholipids), e.g., depending on the size of the structure.

Non-phosphorus containing lipids may also be used such as stearylamine, docecylamine, acetyl palmitate, and fatty acid amides. In other embodiments, other lipids such as fats, oils, waxes, cholesterol, sterols, fat-soluble vitamins (e.g., vitamins A, D, E and K), glycerides (e.g., monoglycerides, diglycerides, triglycerides) can be used to form portions of a structure described herein.

A portion of a structure described herein such as a shell or a surface of a nanostructure may optionally include one or more alkyl groups, e.g., an alkane-, alkene-, or alkyne-containing species that optionally imparts hydrophobicity to the structure. An “alkyl” group refers to a saturated aliphatic group, including a straight-chain alkyl group, branched-chain alkyl group, cycloalkyl (alicyclic) group, alkyl substituted cycloalkyl group, and cycloalkyl substituted alkyl group. The alkyl group may have various carbon numbers, e.g., between C2 and C40, and in some embodiments may be greater than C5, C10, C15, C20, C25, C30, or C35. In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C1-C12 for straight chain, C3-C12 for branched chain), 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclohexyl, and the like.

The alkyl group may include any suitable end group, e.g., a thiol group, an amino group (e.g., an unsubstituted or substituted amine), an amide group, an imine group, a carboxyl group, or a sulfate group, which may, for example, allow attachment of a ligand to a nanostructure core directly or via a linker. For example, where inert metals are used to form a nanostructure core, the alkyl species may include a thiol group to form a metal-thiol bond. In some instances, the alkyl species includes at least a second end group. For example, the species may be bound to a hydrophilic moiety such as polyethylene glycol. In other embodiments, the second end group may be a reactive group that can covalently attach to another functional group. In some instances, the second end group can participate in a ligand/receptor interaction (e.g., biotin/streptavidin).

In some embodiments, the shell includes a polymer. For example, an amphiphilic polymer may be used. The polymer may be a diblock copolymer, a triblock copolymer, etc., e.g., where one block is a hydrophobic polymer and another block is a hydrophilic polymer. For example, the polymer may be a copolymer of an α-hydroxy acid (e.g., lactic acid) and polyethylene glycol. In some cases, a shell includes a hydrophobic polymer, such as polymers that may include certain acrylics, amides and imides, carbonates, dienes, esters, ethers, fluorocarbons, olefins, styrenes, vinyl acetals, vinyl and vinylidene chlorides, vinyl esters, vinyl ethers and ketones, and vinylpyridine and vinylpyrrolidones polymers. In other cases, a shell includes a hydrophilic polymer, such as polymers including certain acrylics, amines, ethers, styrenes, vinyl acids, and vinyl alcohols. The polymer may be charged or uncharged. As noted herein, the particular components of the shell can be chosen so as to impart certain functionality to the structures.

Where a shell includes an amphiphilic material, the material can be arranged in any suitable manner with respect to the nanostructure core and/or with each other. For instance, the amphiphilic material may include a hydrophilic group that points towards the core and a hydrophobic group that extends away from the core, or, the amphiphilic material may include a hydrophobic group that points towards the core and a hydrophilic group that extends away from the core. Bilayers of each configuration can also be formed.

An example of a suitable protein that may associate with a structure described herein is an apolipoprotein, such as apolipoprotein A (e.g., apo A-I, apo A-II, apo A-IV, and apo A-V), apolipoprotein B (e.g., apo B48 and apo B100), apolipoprotein C (e.g., apo C-I, apo C-II, apo C-III, and apo C-IV), and apolipoproteins D, E, and H. Specifically, apo A1, apo A2, and apo E promote transfer of cholesterol and cholesteryl esters to the liver for metabolism and may be useful to include in structures described herein. Additionally or alternatively, a structure described herein may include one or more peptide analogues of an apolipoprotein, such as one described above. A structure may include any suitable number of, e.g., at least 1, 2, 3, 4, 5, 6, or 10, apolipoproteins or analogues thereof. In certain embodiments, a structure includes 1-6 apolipoproteins, similar to a naturally occurring HDL particle. Of course, other proteins (e.g., non-apolipoproteins) can also be included in structures described herein.

It should be understood that the components described herein, such as the lipids, phospholipids, alkyl groups, polymers, proteins, polypeptides, peptides, enzymes, bioactive agents, nucleic acids, and species for targeting described above (which may be optional), may be associated with a structure in any suitable manner and with any suitable portion of the structure, e.g., the core, the shell, or both. For example, one or more such components may be associated with a surface of a core, an interior of a core, an inner surface of a shell, an outer surface of a shell, and/or embedded in a shell. Furthermore, such components can be used, in some embodiments, to facilitate the sequestration, exchange and/or transport of materials (e.g., proteins, peptides, polypeptides, nucleic acids, nutrients) from one or more components of a subject (e.g., cells, tissues, organs, particles, fluids (e.g., blood), and portions thereof) to a structure described herein, and/or from the structure to the one or more components of the subject. In some cases, the components have chemical and/or physical properties that allow favorable interaction (e.g., binding, adsorption, transport) with the one or more materials from the subject.

Combinations

In some embodiments the HDL NP disclosed herein is co-formulated with or administered in conjunction with a ferroptosis inducer. A ferroptosis inducer, as used herein is a compound that initiates, promotes or plays a role in supporting the process of ferroptosis. The HDL NP is administered in conjunction with a compound in any manner in which the compounds are both delivered to a subject. For instance, an HDL NP and compound may be co administered together at the same time or at different times. The two compounds may be administered at the same site or different sites, using the same route of administration or different routes of administrations. In some embodiments the HDL NP may be administered before the compound, such as for instance about 5 minutes, 10 minutes, 30 minutes, 1 hour, 2 hours, 3 hours, 4 hours, 5 hours, 6 hours, 7 hours, 8 hours, 9 hours, 10 hours, 11 hours, 12 hours, 24 hours, 1, 2, 3, 4, 5, 6, or 7 days, 1, 2, 3, 4, 5, 6, 7, 8, 9, or 10 weeks or 1 month, 3 months or 6 months. In other embodiments the HDL NP may be administered after the compound, such as for instance about 5 minutes, 10 minutes, 30 minutes, 1 hour, 2 hours, 3 hours, 4 hours, 5 hours, 6 hours, 7 hours, 8 hours, 9 hours, 10 hours, 11 hours, 12 hours, 24 hours, 1, 2, 3, 4, 5, 6, or 7 days, 1, 2, 3, 4, 5, 6, 7, 8, 9, or 10 weeks or 1 month, 3 months or 6 months. The HDL NP and compound may be administered multiple times in various cycles of administration. Ferroptosis inducer compounds include for instance, compounds that inhibit iron chelation (i.e. iron chelators), compounds which reduce the antioxidant capacity of cells and accumulating ROS, mitochondrial VDAC modulators, modulators of sulfur transfer pathways, and polyunsaturated fatty acids (PUFAs) related compounds such as phosphatidylethanolamine (PE), which contains arachidonic acid (AA) or its derivative adrenaline.

Specific inhibitors of ferroptosis, include for example, ferrostatin-1 (Fer-1), liproxstatin-1, vitamin E, and iron chelators. These substances inhibit ferroptosis typically by inhibiting the formation of lipid peroxides. Fer-1 has been found to inhibit cell death in several in vitro models of diseases such as Huntington's disease (HD), periventricular white matter (PVL), and renal insufficiency. RSL3, DPI7 and DPI10 are ferroptosis inducers, that directly acts on and inhibits the activity of GPX4, also directly act on GPX4 and induce ferroptosis.

Pharmaceutical Compositions

As described herein, the synthetic nanostructures may be used in “pharmaceutical compositions” or “pharmaceutically acceptable” compositions (also referred to as drugs), which comprise a therapeutically effective amount of one or more of the structures described herein, formulated together with one or more pharmaceutically acceptable carriers, additives, and/or diluents. The pharmaceutical compositions described herein may be useful for treating cancer or other conditions. It should be understood that any suitable structures described herein can be used in such pharmaceutical compositions, including those described in connection with the figures. In some cases, the structures in a pharmaceutical composition have a nanostructure core comprising an inorganic material and a shell substantially surrounding and attached to the nanostructure core.

The pharmaceutical compositions may be specially formulated for administration in solid or liquid form, including those adapted for the following: oral administration, for example, drenches (aqueous or non-aqueous solutions or suspensions), tablets, e.g., those targeted for buccal, and sublingual, boluses, powders, granules, pastes for application to the tongue; as a sterile solution or suspension, or sustained-release formulation; spray applied to the oral cavity; for example, as cream or foam.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Pharmaceutical compositions described herein include those suitable for oral administration. The formulations may conveniently be presented in unit dosage form and may be prepared by any methods well known in the art of pharmacy. The amount of active ingredient that can be combined with a carrier material to produce a single dosage form will vary depending upon the host being treated, and the particular mode of administration. The amount of active ingredient that can be combined with a carrier material to produce a single dosage form will generally be that amount of the compound that produces a therapeutic effect. Generally, this amount will range from about 1% to about 99% of active ingredient, from about 5% to about 70%, or from about 10% to about 30%.

The inventive compositions suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a structure described herein as an active ingredient. An inventive structure may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered structure is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the structures described herein include pharmaceutically acceptable emulsions, microemulsions, solutions, dispersions, suspensions, syrups and elixirs. In addition to the inventive structures, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions described herein (e.g., for rectal or vaginal administration) may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the body and release the structures.

The active compound may be mixed under sterile conditions with a pharmaceutically-acceptable carrier, and with any preservatives, buffers, or propellants, which may be required.

The pastes, creams and gels may contain, in addition to the inventive structures, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the structures described herein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Examples of suitable aqueous and nonaqueous carriers, which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the inventive structures may be facilitated by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

Therapeutically Effective Amount

The phrase “therapeutically effective amount” as used herein means that amount of a material or composition comprising an inventive structure that is effective for producing some desired therapeutic effect in a subject at a reasonable benefit/risk ratio applicable to any medical treatment. Accordingly, a therapeutically effective amount may, for example, prevent, minimize, or reverse disease progression associated with a disease or bodily condition. Disease progression can be monitored by clinical observations, laboratory and imaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

An effective amount may depend on the particular condition to be treated. The effective amounts will depend, of course, on factors such as the severity of the condition being treated; individual patient parameters including age, physical condition, size and weight; concurrent treatments; the frequency of treatment; or the mode of administration. These factors are well known to those of ordinary skill in the art and can be addressed with no more than routine experimentation. In some cases, a maximum dose be used, that is, the highest safe dose according to sound medical judgment.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

A physician or veterinarian having ordinary skill in the art can readily determine and prescribe the effective amount of the pharmaceutical composition required. For example, the physician or veterinarian could start doses of the structures described herein employed in the pharmaceutical composition at levels lower than that required to achieve the desired therapeutic effect and then gradually increasing the dosage until the desired effect is achieved.

Subject

As used herein, a “subject” or a “patient” refers to any mammal (e.g., a human), for example, a mammal that may be susceptible to a disease or bodily condition such as the secondary diseases or conditions disclosed herein. Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans. A subject may be a subject diagnosed with a certain disease or bodily condition or otherwise known to have a disease or bodily condition. In some embodiments, a subject may be diagnosed as, or known to be, at risk of developing a disease or bodily condition. In some embodiments, a subject may be diagnosed with, or otherwise known to have, a disease or bodily condition associated with abnormal lipid levels, as described herein. In certain embodiments, a subject may be selected for treatment on the basis of a known disease or bodily condition in the subject. In some embodiments, a subject may be selected for treatment on the basis of a suspected disease or bodily condition in the subject. In some embodiments, the composition may be administered to prevent the development of a disease or bodily condition. However, in some embodiments, the presence of an existing disease or bodily condition may be suspected, but not yet identified, and a composition of the invention may be administered to diagnose or prevent further development of the disease or bodily condition.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

Methods

In some embodiments, the subject has cancer. In some embodiments, the compositions of the instant disclosure (e.g., synthetic nanostructures) may be delivered to a cancer cell (e.g., cell may be contacted) in vitro or ex vivo. The subject may have had cancer in the past and is presently in remission. The subject may presently have an active cancer diagnosis (e.g., is not in remission). The subject may have been diagnosed in any means known in the art to receive the status of having cancer.

In some embodiments, the subject is administered, or the cell is contacted by, any of the compositions described herein (e.g., synthetic nanostructures). The compositions disclosed herein may be administered by any administration route known in the art. For example, in some embodiments, one of ordinary skill in the art, may administer a composition via conventional routes, e.g., administered orally, parenterally, by inhalation spray, topically, rectally, nasally, buccally, vaginally or via an implanted reservoir.

In some embodiments, the subject is administered, or a cell contacted by, a composition (e.g., synthetic nanostructure) at least once. In some embodiments, a subject receives multiple administrations, or a cell is contacted multiple times. For example, without limitation, the subject may receive at least 2 administrations, or a cell contacted at least 2 times (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more). In some embodiments, the administrations or contacts are irregularly spaced (e.g., not an equal duration of time between administrations or contacts). In some embodiments, the administrations or contacts are equally spaced (e.g., an equal duration of time between administrations or contacts). In some embodiments, the subject receives, or the cell is contacted, at least one administration per month. In some embodiments, the subject receives, or the cell is contacted, at least one administration per week. In some embodiments, the subject receives, or the cell is contacted, at least one administration per day. In some embodiments, the subject receives, or the cell is contacted, at least two administrations per day. In some embodiments, where there is more than one administration or contact, the administrations or contacts are of the same route. In some embodiments, where there is more than one administration or contact, the administrations or contacts are of different routes.

### Examples

The data presented in the Examples below demonstrate that gene and protein expression of GPX4 is downregulated after HDL NP exposure, and that this is likely mediated through the high-affinity receptor for cholesterol-rich high-density lipoproteins, SCARB1. RT-qPCR data support that HDL NPs mediate reduced levels of GPX4 by reducing transcription. We have shown that lymphoma cell cholesterol depletion increases the activation of SREBP-1a, which increases de novo cholesterol biosynthesis. SREBP-1a has been reported as a negative regulator of GPX4 expression. Western blot data show a profound reduction in GPX4 which suggest post-translational mechanisms that would further reduce GPX4. Inhibiting de novo cholesterol biosynthesis using statins did not reduce GPX4 expression or induce ferroptosis, suggesting that manipulation of de novo cholesterol biosynthesis is unable to replicate the effects of HDL NP treatment.

Pathways involving intermediates in the cholesterol biosynthesis pathway are interesting in the context of the ALK+ALCL (SR-786, SUDHL1) and U937 cell lines because of their shared inability to synthesize cholesterol due to enzymatic blockade induced by hypermethylation or mutation, respectively. The data presented herein show that HDL NP treatment increased expression of de novo cholesterol synthesis genes and reduced expression of GPX4. In theory, this could serve to even more drastically increase intermediates in the cholesterol biosynthesis pathway that may serve an antioxidant function, but would only be effective at preventing ferroptosis in the presence of GPX4.

The data suggest that investigation of HDL NP in cholesterol auxotrophic cell lines is warranted, despite the fact that these cells can uptake cholesterol via LDLs binding the LDLR. SCARB1 expression was measured in the three auxotrophic cell lines investigated, suggesting that both the LDLR and SCARB1 play a role in supplying the cells with cholesterol. Possible explanations for the observation of potent reduction of GPX4 and ferroptosis after treatment with HDL NP include the following: a) a reduction of cholesterol uptake through LDLR by HDL NP; b) a dependence upon both LDLR and SCARB1 for sufficient cholesterol uptake; or, c) different cellular mechanisms related to cholesterol uptake through HDL via SCARB1 (cell membrane binding) versus LDL via LDLR (particle internalization). In contrast to LDL/LDLR, HDL binding to SCARB1 has been linked to intracellular signaling pathways, including the pro-survival PI3K/AKT pathway. A recent report suggests that a decrease in GPX4 expression correlated with decreased phosphorylation of AKT. Engagement of HDL NPs to SCARB1 may not only prevent cholesterol influx but also disrupts membrane anchored pro-survival signaling pathways that may, ultimately, impact GPX4 expression. Regardless, targeted inhibition of cholesterol uptake by synthetic nanoparticles built upon an inert core appears to be an important target in certain cholesterol auxotrophic or cholesterol uptake dependent cancers.

Interestingly, although the HDL NPs exhibit potent toxicity against the ferroptosis sensitive cancer cells, there is no observed toxicity of normal cells in vitro or in vivo. Based on the data with cancer cells presented herein it is believed that normal cells do not have the same oxidative burden as the cancer cells and the normal cells are able to maintain plasticity with regard to cholesterol metabolism.

### Methods

Cell Lines

The Ramos (RRID: CVCL_0597), SUDHL4 (CVCL_0539), Raji (CVCL_0511), Daudi (CVCL_0008), SUDHL6 (CVCL_2206), Namalwa (CVCL_0067), Jurkat (CVCL_0367), SUDHL1 (CVCL_0538), SR-786 (CVCL_1711), and U937 (CVCL_0007) human cell lines were obtained from ATCC and were used within 3 months of receipt and/or resuscitation. ATCC uses short tandem repeat (STR) profiling to authenticate their cell lines prior to shipping. For SUDHL4 cells, Charles River Laboratories was contracted to test for mycoplasma contamination prior to use in animal experiments. All cell lines were cultured in RPMI 1640 supplemented with 10% fetal bovine serum (FBS) and 1% penicillin/streptomycin at 37° C. in a humidified, 5% CO₂ incubator.

HDL NP Synthesis

The HDL NPs were synthesized and quantified as previously described (36). 5 nm diameter citrate stabilized gold nanoparticles (AuNP) were surface-functionalized with apolipoprotein A-I, followed by addition of the phospholipids, 1,2-dipalmitoyl-sn-glycero-3-phosphoethanolamine-N-[3-(2-pyridyldithio)propionate] (PDP PE) and 1,2-dipalmitoyl-sn-glycero-3-phosphocholine (DPPC). The HDL NPs were purified using the KrosFlo TFF (Tangential Flow Filtration) system with a 50 kDa cut-off PES module. The concentration of HDL NPs was calculated using UV-Vis spectroscopy and Beer's law.

To synthesize fluorescently labeled HDL NPs, the intercalating dye DiI (1,1′-Dioctadecyl-3,3,3′,3′-tetramethylindocarbocyanine perchlorate) was added at a 1 μM final concentration during the phospholipid addition step. Purification and quantification of the fluorescently labeled HDL NPs was conducted, as described above.

HDL NP Binding to SCARB1 Assay

Ramos, SUDHL4, and Jurkat cells were incubated with DiI HDL NPs (10 nM) in standard culture media for 2 hours at 37° C., in the presence or absence of the SCARB1 blocking antibody (Novus Biologicals; 1:100; RRID: AB_1291690), and/or the rabbit IgG isotype control antibody (Novus Biologicals; 1:100). Cells were washed once with 1 mL of ice-cold FACS buffer (PBS, 1% bovine serum albumin, 0.1% sodium azide) and re-suspended in 500 μl of ice-cold FACS buffer prior to flow cytometric analysis (BD LSR II Fortessa). Data were analyzed using the FCS Express software.

Western Blot Analysis

Western blots were conducted as previously described. Blots were imaged using the Azure 3000 imager. The SCARB1 antibody (Abeam, RRID: AB_882458; 1:1,000), the GPX4 antibody (Abeam, AB_941790; 1:5,000), the β-actin antibody (Cell Signaling Technologies, AB_2223172; 1:3,000) and a secondary antibody (goat anti-rabbit HRP, BioRad, AB_111251.42; 1:2,000) were used in these experiments.

RT-qPCR Analysis

Ramos, SUDHL4, SUDHL1, SR-786 and U937 cells were treated with HDL NPs (20 nM, 50 nM), human HDL (hHDL; 50 nM) or PBS for up to 72 hours, and RNA isolated using the RNeasy Mini kit (Qiagen). In all cases, hHDL was added at an equimolar concentration to HDL NPs based upon protein concentration). RNA samples (500 ng RNA/30 μl reaction) were reverse transcribed using a TaqMan Reverse Transcription kit, and qPCR was performed using Taqman Gene Expression Assays (Life Technologies) on a BioRad (TX-Connect iCycler. Samples were standardized to β-actin, and relative expression was calculated using the ΔΔCt method. Biological triplicates were run for each condition.

C11-BODIPY Assay for Lipid Peroxidation

Ramos, SUDHL4, SUDHL1, SR-786 and U937 cells (2.5×10⁵ cells/ml) were treated with HDL NPs (50 nM) or PBS for 24, 48 or 72 hours. Following treatment, C11-BODIPY (1 μM final concentration; Thermo Fisher Scientific) was added to each well and the cells were incubated for 30 minutes at 37° C., 5% CO₂. The cells were then washed twice with 1×PBS, re-suspended in ice-cold FACS buffer and C11-BODIPY fluorescence in the FITC channel quantified using the BD LSR II Fortessa flow cytometer. Data were analyzed using the FCS Express software.

Cell Death (MTS) Assay

MTS assays (CellTiter; Promega) were conducted as described previously. For the Ramos, SUDHL4, Raji, Daudi, Namalwa, SUDHL6 and Jurkat cells were plated at a density of 2×10⁵ cells/mL, and cultured for 72 hours prior to assay. SUDHL1, SR-786, and U937 cells were plated at a density of 5×10⁴ cells/mL and cultured for 5 days prior to MTS assay. The SCARB1 blocking and isotype control antibodies were added at a dilution of 1:1000 to 1:250. Ferrostatin-1 and deferoxamine (DFO) were obtained from Sigma Aldrich, and added at a final concentration of 1 μM. MTS values were standardized to PBS control.

Tumor Xenograft Model

SCID-beige mice (4 to 6 weeks old; Charles River) were used for the SUDHL4 tumor xenograft study. Flank tumors were initiated using 1×10⁷ SUDHL4 cells per mouse. Tumors were allowed to reach ˜100 mm³ before HDL NP treatments began. Based on their initial tumor volumes, mice were randomly divided into 2 groups, PBS (100 μL) and HDL NPs (100 μL of 1 μM NPs). Treatments (intravenous) were administered 3 times per week for 1 week. Tumors were then harvested, and single cell suspensions generated by mechanically dissociating the tumors and passing the cells through a 70-micron filter. C11-BODIPY (1 μM final concentration) was added to a fraction of the resultant cell suspension (1×10⁶ cells) and flow analysis was carried out as described above. RNA was isolated from the remainder of the cells to quantify GPX4 expression by RT-qPCR, as described above.

Human Tissue Analysis

Archived, formalin-fixed, paraffin embedded tissue sections were analyzed from patients with large B cell lymphoma and follicular lymphoma. All samples were de-identified of all information other than final diagnosis. A total of 104 follicular lymphoma and 49 diffuse large B cell lymphoma archival samples were obtained and stained for SCARB1 expression. Immunohistochemical staining of the sections was performed using a monoclonal SCARB1 antibody (Abeam, AB_882458; 1:100 dilution) by the Pathology Core at the Robert H. Lurie Comprehensive Cancer Center of Northwestern University. Liver and thymus specimens were utilized as positive and negative controls, respectively. Bright field images were captured at 10× and 40× magnifications.

### Results

HDL NPs Down-Regulate GPX4

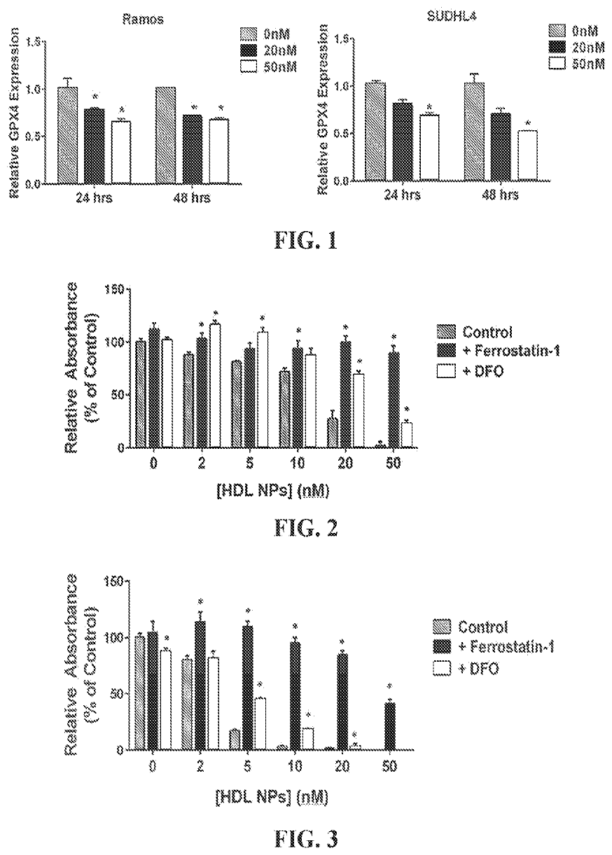

A study was performed to determine whether HDL NPs increase expression of de novo cholesterol synthesis genes and reduce expression of GPX4. The data is shown in FIG. 1. Ramos and SUDHL4 cells, are well studied models of Burkitt's lymphoma (BL) and germinal center DLBCL (GC DLBCL) respectively. HDL NPs cause cellular cholesterol depletion and profound in vitro and in vivo cell death in SUDHL4 and Ramos cells. RT-qPCR analysis was performed to assess GPX4 expression in Ramos (left panel) and SUDHL4 (right panel) cells treated with HDL NPs for 24 or 48 hours at 0 nM (control), 20 nM, or 50 nM. Decreased GPX4 expression was confirmed using western blot analysis and conventional RT-qPCR. It was observed that HDL NP treatment profoundly reduced expression of GPX4 in both cell lines relative to PBS control (0 nM) at both the protein (not shown) and mRNA level (FIG. 1). By contrast, treatment with an equimolar concentration of cholesterol-rich HDL did not alter GPX4 protein or gene expression (not shown).

HDL NP Induces Ferroptosis in B Cell Lymphoma Cell Lines

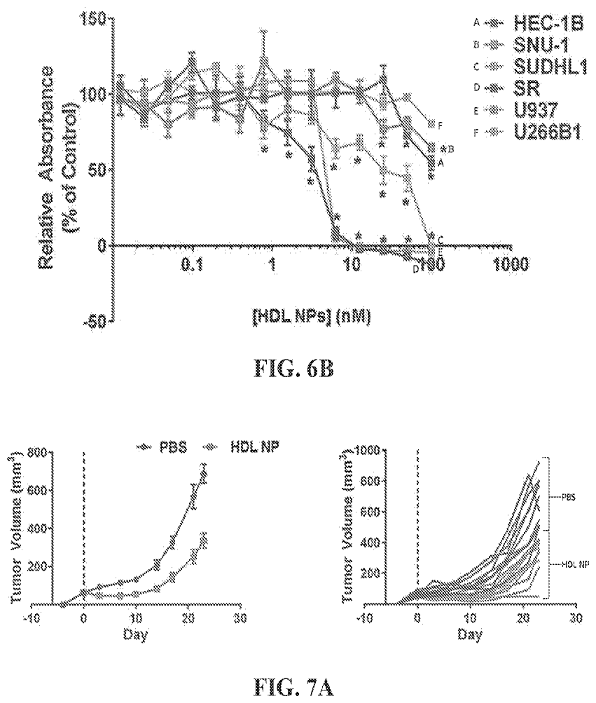

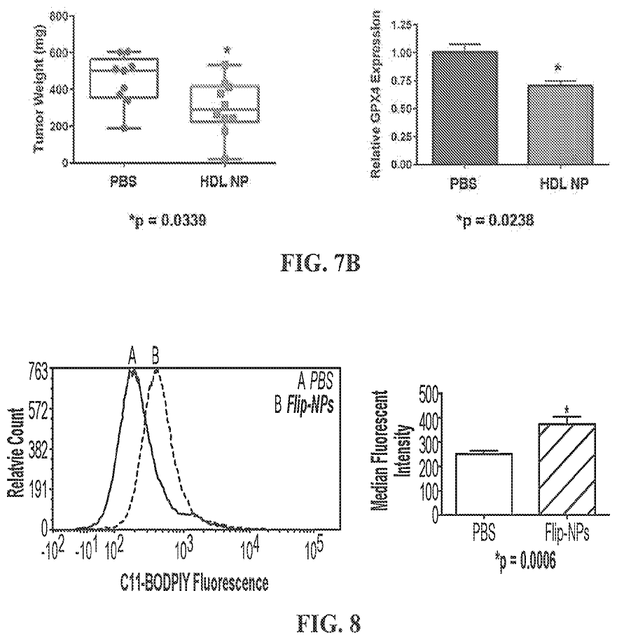

At least two metrics have been proposed to distinguish ferroptosis from apoptosis and other forms of cell death: 1) Cell death correlates with an increase in oxidized membrane lipids quantified by using C11-BODIPY, a lipophilic fluorescent dye that has a unique spectral signature when oxidized and is used to measure lipid peroxidation, and flow cytometry; and 2) Cell death can be reduced by addition of a lipophilic antioxidant (e.g. ferrostatin-1) or an iron chelator, e.g. deferoxamine (DFO). Using these metrics, whether HDL NPs induced ferroptosis in Ramos and SUDHL4 cells was assessed. In both cell lines, the HDL NP treatment led to a dose-dependent increase in C11-BODIPY signal over time. Whether HDL NPs induce lipid peroxide accumulation in SUDHL4 cells was assessed and the data is shown in FIG. 4A-4B. Similarly whether HDL NPs induce lipid peroxide accumulation in Ramos cells was assessed and the data is shown in FIG. 5A-5B. The dose dependent increase of lipid peroxide accumulation in both cell lines was confirmed.

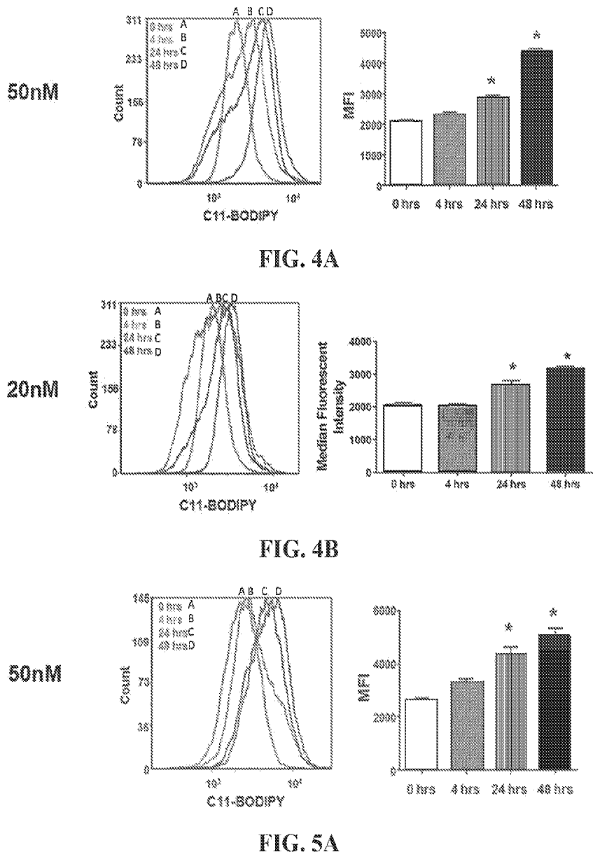

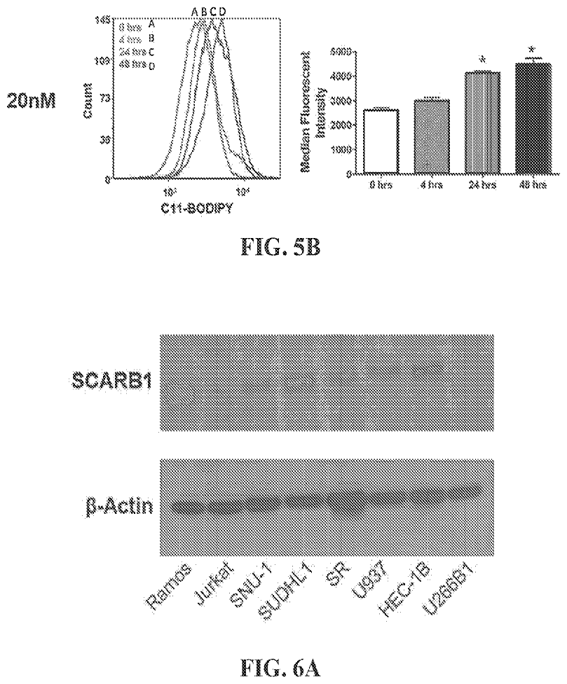

Ramos and SUDHL4 cells were then cultured with HDL NPs in the presence of either ferrostatin-1 or DFO and assayed for cell viability. Addition of ferrostatin-1 and DFO significantly inhibited HDL NP induced cell death in SUDHL4 (Diffuse Large B Cell Lymphoma, FIG. 2) and Ramos (Burkitt's Lymphoma, FIG. 3) cells. These data demonstrate that HDL NPs induce ferroptosis in Ramos and SUDHL4 cells.

HDL NP Induces Ferroptosis in Cholesterol Auxotrophic Lymphoma Cell Lines

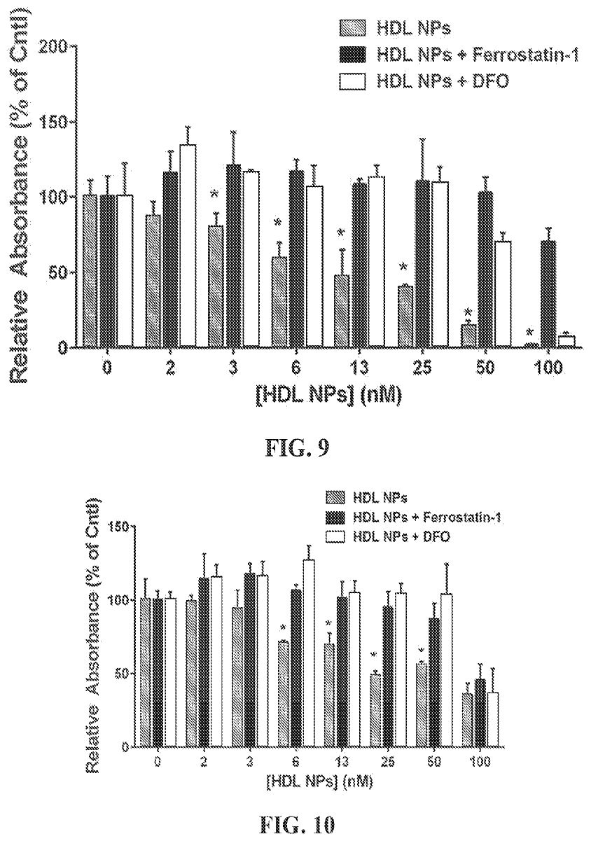

A number of cell lines are auxotrophic for cholesterol, including the cell lines SR-786 (ALK+ALCL), SUDHL1 (ALK+ALCL), and U937 (isolated from histiocytic lymphoma, but of myeloid lineage), among others. The ALK+ALCL cells were identified based upon reduced viability when cultured in lipoprotein deficient serum, and the cell death phenotype was rescued by addition of cholesterol-rich low-density lipoprotein (LDL) or free cholesterol. HDL NPs target SCARB1 in SUDHL4 and Ramos cells, resulting in cellular cholesterol depletion and profound in vitro and in vivo cell death. The requirement of SCARB1 as a target of HDL NP in these lymphoma cells using an anti-SCARB1 blocking antibody and fluorescently labeled HDL NPs was verified (data not shown). The expression of SCARB1 in ALK+ALCL and U937 cells was also examined. Data reveal SCARB1 expression in SR-786, SUDHL1 and U937 cells (FIG. 6A). Treatment of each of the cell lines with HDL NPs potently induced cell death (FIG. 6B). The data in FIG. 6 show the effect of SR-B1 expression and HDL NP efficacy in cholesterol auxotrophic cell lines. The tested cells include SNU-1: Gastric cancer. SUDHL1: ALK+Anaplastic Large T Cell Lymphoma. SR: ALK+Anaplastic Large T Cell Lymphoma. U937: Histiocytic lymphoma. HEC-1B: Endometrial adenocarcinoma. U266B1: Myeloma. Ramos and Jurkat represent positive and negative controls for SCARB1 expression, respectively. β-actin was used as a loading control. The cells were treated with HDL NPs for 120 hours.

HDL NP Induces Ferroptosis In Vivo

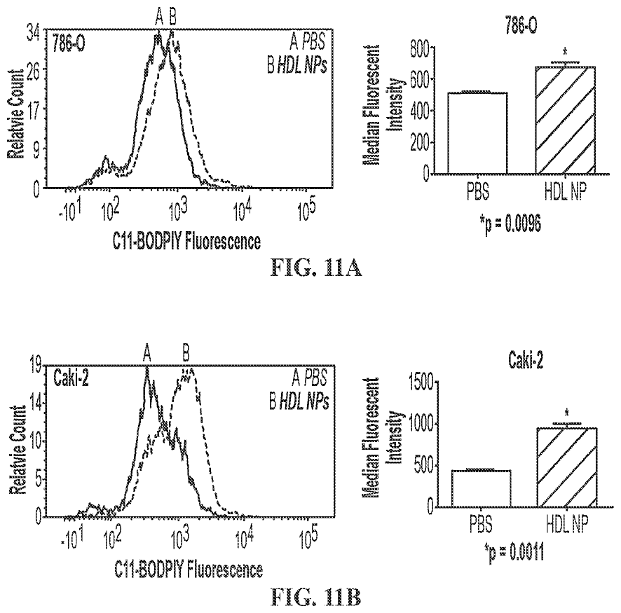

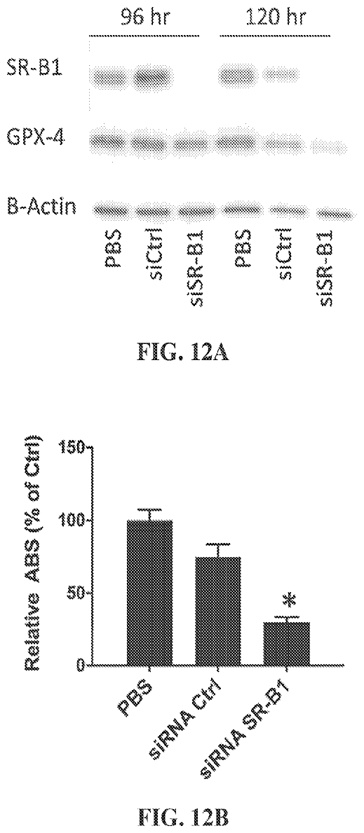

HDL NPs specifically target and significantly reduce tumor burden in xenograft models as shown in SUDHL4 and Ramos cells. To determine if systemic HDL NP treatment reduces GPX4 expression and increases lipid peroxide accumulation in tumor cells in vivo, SUDHL4 tumor xenografts (˜100 mm³ in volume) were established in SCID-beige mice. The mice were then treated with PBS or HDL NPs (100 μl of 1 μM HDL NP, 3 times per week for 1 week, i.v.). Following treatment, tumors were resected and GPX4 expression and lipid peroxide accumulation were quantified by RT-qPCR and C11-BODIPY staining, respectively. HDL NP treatment led to a down-regulation of GPX4 as measured by RT-qPCR compared with PBS controls (FIG. 7B), which correlated with an increase in membrane lipid peroxide accumulation. The changes in tumor volume are shown in FIG. 7A. No adverse side effects were observed after systemic administration of HDL NPs. These data show that HDL NPs induce molecular changes consistent with ferroptosis in the SUDHL4 flank tumor xenograft model of lymphoma. The lipid accumulation in SUDHL1 cells in the in vivo Ferroptosis assay is shown in the plots of FIG. 8.

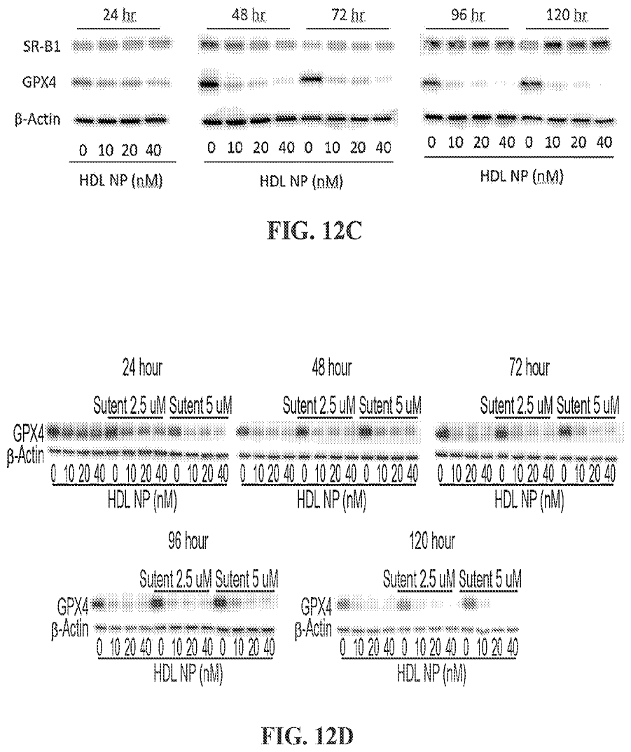

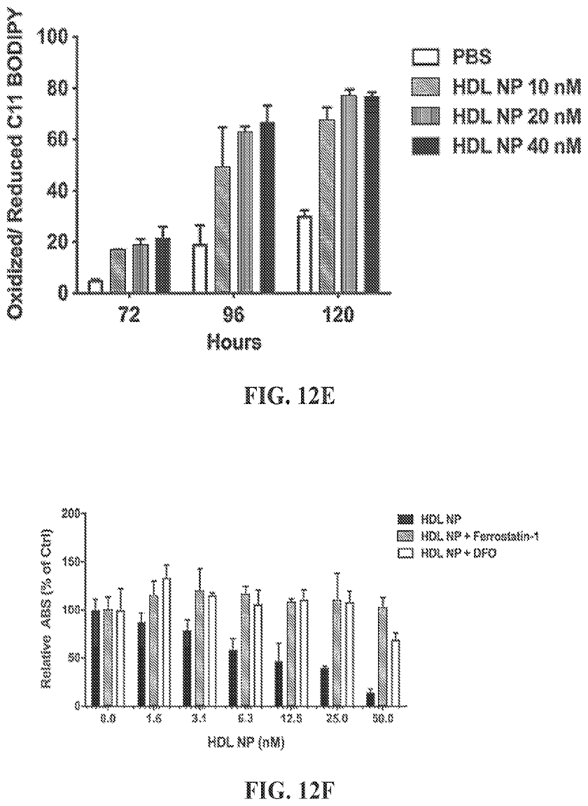

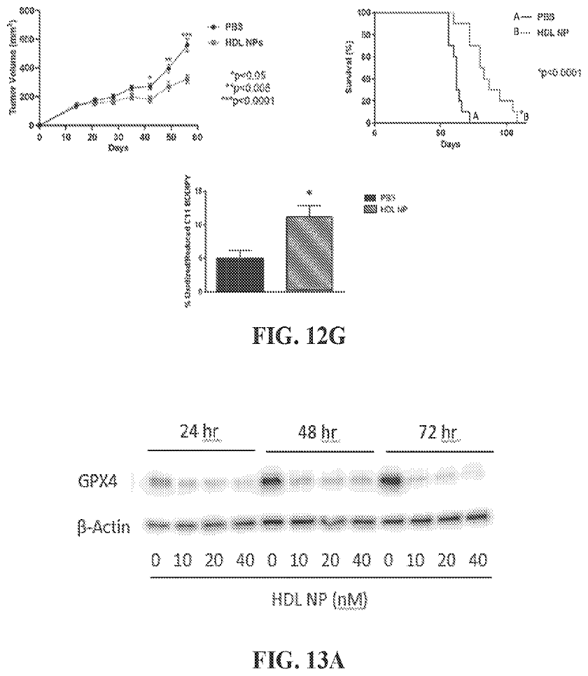

Studies extending these findings to renal cell carcinomas are shown in FIGS. 9-11. FIG. 9 is a bar graph showing that HDL NPs induce Ferroptosis in 786-O (renal cell carcinoma-clear cell) cells (HDL NPs+Ferrostatin-1 and HDL NPs+DFO). The Ferrostatin-1 had dramatic effects on the HDL NP function, particularly at higher concentrations. The data of FIG. 10 show that HDL NPs also induce Ferroptosis in Caki-2 (renal cell carcinoma-papillary) cells. Similarly, HDL NPs induce lipid peroxide accumulation in 786-O and Caki-2 cells (FIGS. 11A-11B).

GPX4 and SR-B1 Expression in response to HDL NP in Renal Cell Carcinoma and Ovarian Cancer Cell Lines

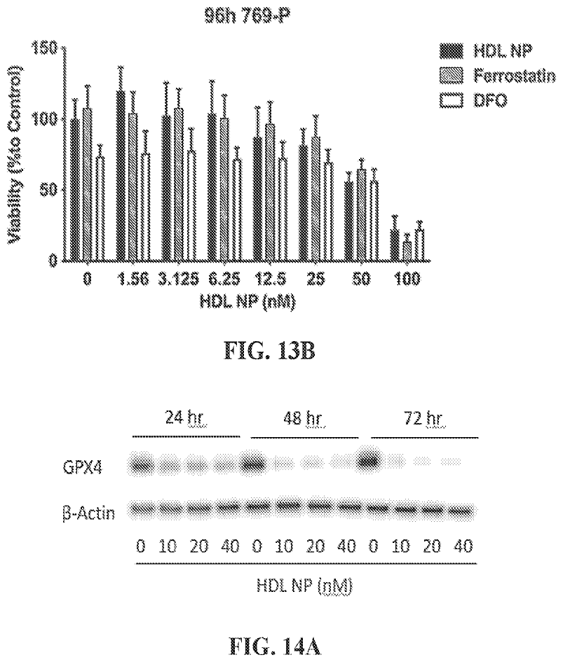

The role of SR-B1 in GPX4 expression was analyzed using siRNA. Specific knockdown of SR-B1 using siRNA was shown to downregulate GPX4 expression. Following treatment with SR-B1 siRNA, an siRNA control (siCtrl) or PBS at 96 and 120 hours (hr) protein was isolated and examined by Western Blot (25 μg of protein, SR-B1 antibody Abcam (ab52629, 1:2,000), GPX4 Abcam (ab41787, 1:20,000)) Beta actin Cell Signaling (13E5, 1:2,000) served as a protein control. Data is shown in FIG. 12A, demonstrating complete loss of protein expression with SR-B1 knockdown. The data of FIG. 12B shows that siRNA knockdown of SR-B1 downregulation induces cell death.

The renal cell carcinoma cell line was further treated with HDL NP to assess the impact of HDL NP on SR-B1 and GPX4 expression. Western Blot analysis of GPX4 and SR-B1 over various time courses with varying concentrations of HDL NPs was performed. The data is shown in FIG. 12C. The data demonstrate that HDL NPs do not directly regulate SR-B1 receptor expression; however, HDL NPs drastically downregulate GPX4 expression in a time and dose (e.g., HDL NP concentration) dependent manner.

The ability of HDL NP to impact GPX4 expression in the presence of Sutent, a targeted receptor protein-tyrosine kinase inhibitor therapy was also examined. The results are shown in FIG. 12D, which illustrates that HDL NPs drastically downregulate GPX4 expression in the presence of Sutent. 8 μg protein, GPX4 Abcam (ab41787, 1:5,000), Beta actin Cell Signaling (13E5, 1:2,000) were used. The HDL NPs were shown to also increase the expression of oxidized lipids (FIG. 12E). Using an MTS Rescue Assay; cell death induced by HDL NPs was found to be rescued by ferrostatin-1 and deferoxamine (FIG. 12F). FIG. 12G shows in vivo data of HDL NPs reducing 786-O tumor burden (upper left panel); HDL NPs increase survival (upper right panel); and HDL NPs increase oxidized lipids in tumors after 5 treatments of the HDL NP (bottom middle panel).

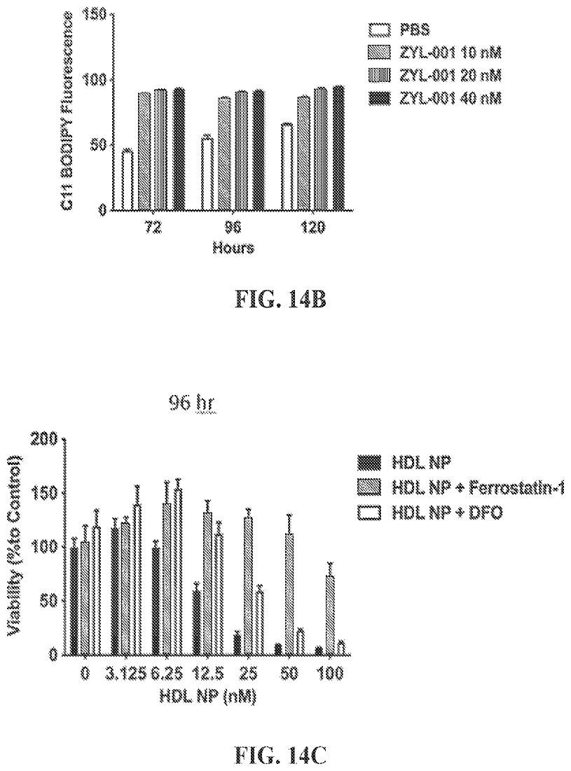

The impact of HDL NPs in another renal cell carcinoma cell line, 769-P was further examined. The results shown in FIGS. 13A-13B show that HDL NPs also downregulate GPX4 expression in these cells (FIG. 13A, Western Blot analysis) and induce cell death which is rescued by ferrostatin-1 and deferoxamine (FIG. 13B MTS data).

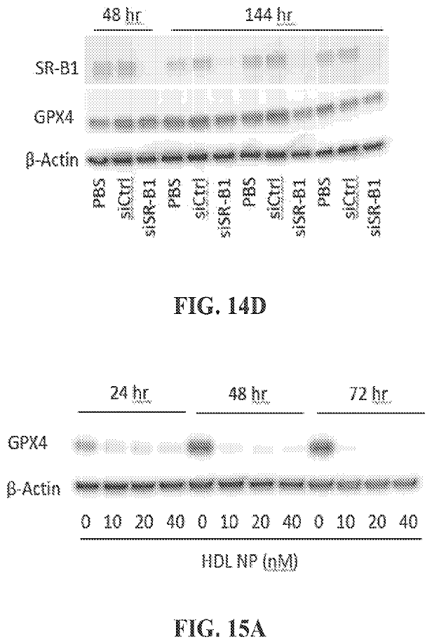

Similar experiments were carried out in ovarian cancer cells, with consistent data, which is presented in FIGS. 14-16, FIGS. 14A-14D shows results generated from HDL NPs in OVCAR5 cell line, a platinum sensitive ovarian cancer cell line. A Western Blot of GPX4 illustrating HDL NPs downregulate GPX4 expression is shown in FIG. 14A. C11-BODIPY Flow Data illustrating that HDL NPs increase the expression of oxidized lipids is shown in FIG. 14B. The results of an MTS assay showing cell death induced by HDL NPs is rescued by ferrostatin-1 and deferoxamine is presented in FIG. 14C. FIG. 14D shows Western Blots of SR-B1 and GPX4 and that siRNA knockdown of SR-B1 downregulates GPX4 expression.

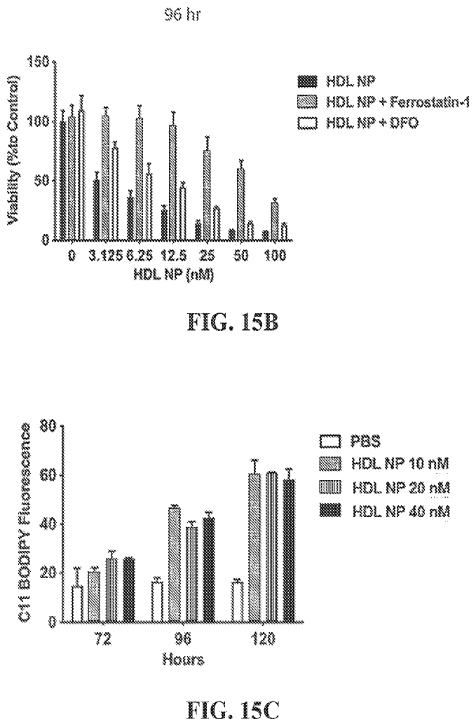

An OVCAR5 CP Resistant Cell Line, a platinum resistant ovarian cancer cell line was used to generate similar data, which is presented in FIGS. 15A-15C. FIG. 15A shows a Western Blot of GPX4 and that HDL NPs downregulate GPX4 expression. FIG. 15B shows an MTS assay of cell death induced by HDL NPs is rescued by ferrostatin-1 and deferoxamine. FIG. 15C shows HDL NPs increase the expression of oxidized lipids.

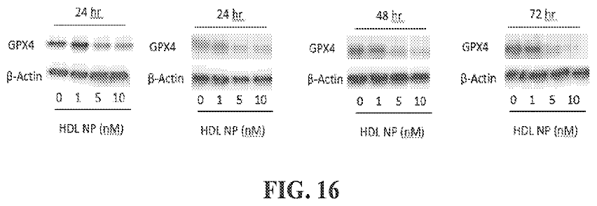

Similarly the ES2 Cell Line, a clear cell ovarian carcinoma cell line was used to generate similar data and the results are shown in FIG. 16. Western Blot are shown of GPX4 and that HDL NPs downregulate GPX4 expression.

### Other Embodiments

Embodiment 1. A method of treating a subject having, suspected of having, or at risk of having cancer comprising: administering to the subject a synthetic nanostructure comprising: a nanostructure core, a shell comprising a lipid surrounding and attached to the nanostructure core, wherein the shell comprises a phospholipid; wherein the subject has cancer cells and wherein the synthetic nanostructure is administered in an effective amount to induce ferroptosis in the cancer cells.

Embodiment 2. A method of reducing, in a population of cells, the number of cancer cells, the method comprising: contacting the cancer cells with a synthetic nanostructure comprising: a nanostructure core, a shell comprising a lipid surrounding and attached to the nanostructure core, wherein the shell comprises a phospholipid; wherein the synthetic nanostructure is in an effective amount to induce ferroptosis in the cancer cells.

Embodiment 3. The method of any one of embodiments 1-2, wherein the nanostructure core is gold.

Embodiment 4. The method of any one of embodiments 1-3, wherein the synthetic nanostructure further comprises an apolipoprotein.

Embodiment 5. The method of embodiment 1-4, wherein apolipoprotein is apolipoprotein A-I, apolipoprotein A-II, or apolipoprotein E.

Embodiment 6. The method of any one of embodiments 1-5, wherein the synthetic nanostructure further comprises a cholesterol.

Embodiment 7. The method of any one of embodiments 1-6, wherein the phospholipid shell comprises a lipid monolayer.

Embodiment 8. The method of any one of embodiments 1-6, wherein the phospholipid shell comprises a lipid bilayer.

Embodiment 9. The method of embodiment 8, wherein at least a portion of the lipid bilayer is covalently bound to the nanostructure core.

Embodiment 10. The method of any one of embodiments 1-9, wherein the nanostructure core has a largest cross-sectional dimension of less than or equal to about 500 nanometers (nm).

Embodiment 11. The method of any one of embodiments 1-10, wherein the nanostructure core has a largest cross-sectional dimension of less than or equal to about 250 nanometers (nm).

Embodiment 12. The method of any one of embodiments 1-11, wherein the nanostructure core has a largest cross-sectional dimension of less than or equal to about 100 nanometers (nm).

Embodiment 13. The method of any one of embodiments 1-12, wherein the nanostructure core has a largest cross-sectional dimension of less than or equal to about 75 nanometers (nm).

Embodiment 14. The method of any one of embodiments 1-13, wherein the nanostructure core has a largest cross-sectional dimension of less than or equal to about 50 nanometers (nm).

Embodiment 15. The method of any one of embodiments 1-14, wherein the nanostructure core has a largest cross-sectional dimension of less than or equal to about 30 nanometers (nm).

Embodiment 16. The method of any one of embodiments 1-15, wherein the nanostructure core has a largest cross-sectional dimension of less than or equal to about 15 nanometers (nm).

Embodiment 17. The method of any one of embodiments 1-16, wherein the nanostructure core has a largest cross-sectional dimension of less than or equal to about 10 nanometers (nm).

Embodiment 18. The method of any one of embodiments 1-17, wherein the nanostructure core has a largest cross-sectional dimension of less than or equal to about 5 nanometers (nm).

Embodiment 19. The method of any one of embodiments 1-18, wherein the nanostructure core has a largest cross-sectional dimension of less than or equal to about 3 nanometers (nm).

Embodiment 20. The method of any one of embodiments 1-19, wherein the nanostructure core has an aspect ratio of greater than about 1:1.

Embodiment 21. The method of any one of embodiments 1-20, wherein the nanostructure core has an aspect ratio of greater than 3:1.

Embodiment 22. The method of any one of embodiments 1-21, wherein the nanostructure core has an aspect ratio of greater than 5:1.

Embodiment 23. The method of any one of embodiments 1-22, wherein the phospholipid comprise 1,2-dipalmitoyl-sn-glycero-3-phosphocholine (DPPC), 1,2-dioleoyl-sn-glycero-3-phosphocholine (DOPC), 1,2-dipalmitoyl-sn-glycero-3-phosphoethanolamine (16:0 PE), 1,2-distearoyl-sn-glycero-3-phosphoethanolamine (18:0 PE), sphingomyelin, 1,2-dioleoyl-3-trimethylammonium-propane (DOTAP), or a combination thereof.

Embodiment 24. The method of embodiment 1-23, wherein the subject has been diagnosed with cancer.

Embodiment 25. The method of any one of embodiments 1-24, wherein the subject has been diagnosed with a ferroptosis sensitive malignancy or cholesterol auxotrophic malignancy.

Embodiment 26. The method of any one of embodiments 1-25, wherein the cancer is selected from: B-cell lymphoma, renal cell carcinoma, T-cell lymphoma, gastric cancer, ovarian carcinoma, and endometrial adenocarcinoma.

Embodiment 27. The method of any one of embodiments 1-26, wherein the cancer is selected from: sarcoma, lymphoma, gastric cancer, anaplastic large cell lymphoma, clear cell renal cell carcinoma (ccRCC), ovarian cancer, platinum resistant ovarian cancer, and clear cell ovarian cancer.

Embodiment 28. The method of any one of embodiments 1-27, wherein the synthetic nanostructure is administered to the subject or contacted to the cells more than once.

Embodiment 29. The method of embodiment 28, wherein the synthetic nanostructure is administered to the subject or contacted to the cells at least once per month.

Embodiment 30. The method of any one of embodiments 28-29, wherein the synthetic nanostructure is administered to the subject or contacted to the cells at least once per week.

Embodiment 31. The method of any one of embodiments 28-30, wherein the synthetic nanostructure is administered to the subject or contacted to the cells at least once per day.

Embodiment 32. The method of any one of embodiments 28-31, wherein the synthetic nanostructure is administered to the subject or contacted to the cells twice per day.

Embodiment 33. The method of any one of 1-32, wherein the subject is a mammal.

Embodiment 34. The method of any one of embodiments 1-33, wherein the subject is human.

Embodiment 35. The method of any one of embodiments 1-33, further comprising administering to the subject a ferroptosis inducer compound.

Embodiment 36. The method of any one of embodiments 1-33, further comprising determining if the cancer is sensitive to ferroptosis.

Embodiment 37. A method of treating a subject having a ferroptosis sensitive disorder comprising: identifying a subject having a ferroptosis sensitive disorder; and administering to the subject a synthetic nanostructure comprising: a nanostructure core, a shell comprising a lipid surrounding and attached to the nanostructure core, wherein the shell comprises a phospholipid; in an effective amount to induce ferroptosis in diseased cells of the subject.

Embodiment 38. A composition comprising synthetic nanostructure comprising: a nanostructure core, a shell comprising a lipid surrounding and attached to the nanostructure core, wherein the shell comprises a phospholipid and a ferroptosis inducer compound.

Embodiment 39. A method for inducing ferroptosis in a cell, comprising: identifying a cell as being a ferroptosis sensitive cell, and contacting the cell with a nanostructure core, a shell comprising a lipid surrounding and attached to the nanostructure core, wherein the shell comprises a phospholipid in an effective amount to induce ferroptosis in the cell.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method of treating a subject having cancer comprising:
identifying a subject having a ferroptosis sensitive malignancy,
administering to the subject a synthetic nanostructure comprising:
a nanostructure core, a shell comprising a lipid surrounding and attached to the nanostructure core, wherein the shell comprises a phospholipid;
wherein the subject has cancer cells and wherein the synthetic nanostructure is administered in an effective amount to induce ferroptosis in the cancer cells.

2. A method of reducing, in a population of cells, the number of cancer cells, the method comprising:
contacting the cancer cells with a synthetic nanostructure comprising:
a nanostructure core, a shell comprising a lipid surrounding and attached to the nanostructure core, wherein the shell comprises a phospholipid;
wherein the synthetic nanostructure is in an effective amount to induce ferroptosis in the cancer cells.

3. The method of any one of claims 1-2, wherein the nanostructure core is gold.

4. The method of any one of claims 1-3, wherein the synthetic nanostructure further comprises an apolipoprotein.

5. The method of any one of claims 1-4, wherein apolipoprotein is apolipoprotein A-I, apolipoprotein A-II, or apolipoprotein E.

6. The method of any one of claims 1-5, wherein the synthetic nanostructure further comprises a cholesterol.

7. The method of any one of claims 1-6, wherein the phospholipid shell comprises a lipid monolayer.

8. The method of any one of claims 1-6, wherein the phospholipid shell comprises a lipid bilayer.

9. The method of claim 8, wherein at least a portion of the lipid bilayer is covalently bound to the nanostructure core.

10. The method of any one of claims 1-9, wherein the nanostructure core has a largest cross-sectional dimension of less than or equal to about 500 nanometers (nm).

11. The method of any one of claims 1-10, wherein the nanostructure core has a largest cross-sectional dimension of less than or equal to about 250 nanometers (nm).

12. The method of any one of claims 1-11, wherein the nanostructure core has a largest cross-sectional dimension of less than or equal to about 100 nanometers (nm).

13. The method of any one of claims 1-12, wherein the nanostructure core has a largest cross-sectional dimension of less than or equal to about 75 nanometers (nm).

14. The method of any one of claims 1-13, wherein the nanostructure core has a largest cross-sectional dimension of less than or equal to about 50 nanometers (nm).

15. The method of any one of claims 1-14, wherein the nanostructure core has a largest cross-sectional dimension of less than or equal to about 30 nanometers (nm).

16. The method of any one of claims 1-15, wherein the nanostructure core has a largest cross-sectional dimension of less than or equal to about 15 nanometers (nm).

17. The method of any one of claims 1-16, wherein the nanostructure core has a largest cross-sectional dimension of less than or equal to about 10 nanometers (nm).

18. The method of any one of claims 1-17, wherein the nanostructure core has a largest cross-sectional dimension of less than or equal to about 5 nanometers (nm).

19. The method of any one of claims 1-18, wherein the nanostructure core has a largest cross-sectional dimension of less than or equal to about 3 nanometers (nm).

20. The method of any one of claims 1-19, wherein the nanostructure core has an aspect ratio of greater than about 1:1.

21. The method of any one of claims 1-20, wherein the nanostructure core has an aspect ratio of greater than 3:1.

22. The method of any one of claims 1-21, wherein the nanostructure core has an aspect ratio of greater than 5:1.

23. The method of any one of claims 1-22, wherein the phospholipid comprise 1,2-dipalmitoyl-sn-glycero-3-phosphocholine (DPPC), 1,2-dioleoyl-sn-glycero-3-phosphocholine (DOPC), 1,2-dipalmitoyl-sn-glycero-3-phosphoethanolamine (16:0 PE), 1,2-distearoyl-sn-glycero-3-phosphoethanolamine (18:0 PE), sphingomyelin, 1,2-dioleoyl-3-trimethylammonium-propane (DOTAP), or a combination thereof.

24. The method of any one of claims 1-23, wherein the subject has been diagnosed with cancer.

25. The method of any one of claims 1-24, wherein the subject has been diagnosed with a ferroptosis sensitive malignancy or cholesterol auxotrophic malignancy.

26. The method of any one of claims 1-25, wherein the cancer is selected from: B-cell lymphoma, renal cell carcinoma, T-cell lymphoma, gastric cancer, ovarian carcinoma, and endometrial adenocarcinoma.

27. The method of any one of claims 1-26, wherein the cancer is selected from: sarcoma, lymphoma, gastric cancer, anaplastic large cell lymphoma, clear cell renal cell carcinoma (ccRCC), ovarian cancer, platinum resistant ovarian cancer, and clear cell ovarian cancer B-cell lymphoma and T-cell lymphoma.

28. The method of any one of claims 1-27, wherein the synthetic nanostructure is administered to the subject or contacted to the cells more than once.

29. The method of claim 28, wherein the synthetic nanostructure is administered to the subject or contacted to the cells at least once per month.

30. The method of any one of claims 28-29, wherein the synthetic nanostructure is administered to the subject or contacted to the cells at least once per week.

31. The method of any one of claims 28-30, wherein the synthetic nanostructure is administered to the subject or contacted to the cells at least once per day.

32. The method of any one of claims 28-31, wherein the synthetic nanostructure is administered to the subject or contacted to the cells twice per day.

33. The method of any one of claims 1-32, wherein the subject is a mammal.

34. The method of any one of claims 1-33, wherein the subject is human.

35. The method of any one of claims 1-33, further comprising administering to the subject a ferroptosis inducer compound.

36. The method of any one of claims 1-33, further comprising determining if the cancer is sensitive to ferroptosis.

37. A method of treating a subject having a ferroptosis sensitive disorder comprising:
identifying a subject having a ferroptosis sensitive disorder; and
administering to the subject a synthetic nanostructure comprising:
a nanostructure core, a shell comprising a lipid surrounding and attached to the nanostructure core, wherein the shell comprises a phospholipid; in an effective amount to induce ferroptosis in diseased cells of the subject.

38. A composition comprising synthetic nanostructure comprising:
a nanostructure core, a shell comprising a lipid surrounding and attached to the nanostructure core, wherein the shell comprises a phospholipid and a ferroptosis inducer compound.

39. A method for inducing ferroptosis in a cell, comprising:
identifying a cell as being a ferroptosis sensitive cell, and contacting the cell with a nanostructure core, a shell comprising a lipid surrounding and attached to the nanostructure core, wherein the shell comprises a phospholipid in an effective amount to induce ferroptosis in the cell.
